(19) United States
(12) Patent Application Publication
(10) Pub. No.: US 2025/0177012 A1
(43) Pub. Date: Jun. 5, 2025

(54) SPINAL LOCKING PLATE FOR CERVICAL SPINE FIXATION

(71) Applicant: Dimitriy KONDRASHOV, San Francisco, CA (US)

(72) Inventor: Dimitriy KONDRASHOV, San Francisco, CA (US)

(21) Appl. No.: 18/842,568

(22) PCT Filed: Mar. 10, 2023
(86) PCT No.: PCT/US2023/015001

§ 371 Date: Aug. 29, 2024

Related U.S. Application Data
(60) Provisional application No. 63/319,628, filed on Mar. 14, 2022.

(51) Int. Cl. A61B 17/70

(52) U.S. Cl. CPC A61B 17/7059

(57) ABSTRACT

Disclosed herein is a device that comprises include a plate; a plurality of locking screws attached to the plate; a locking polyaxial and/or a monoaxial screw; a tulip assembly, having inner threads and a bottom hole, attached to the plate with the plurality of the locking polyaxial and/or monoaxial screw; a set screw having threads; and a rod connected to the tulip assembly by the set screw.

### FIELD OF THE INVENTION

The present invention relates generally to a spinal plate for spinal fixation during a spinal fusion surgery. More specifically, the present invention is a device that provides stronger fixation in patients while reducing risk of nerve and vascular damage during cervical, thoracic, lumbar and sacral spine surgery.

### BACKGROUND OF THE INVENTION

Spinal fusion is a traditional treatment of various diseases affecting the cervical spine. The most common pathology requiring a cervical fusion is compression of cervical spinal cord, compression of nerve roots—radiculopathy, instability—spondylolisthesis, cervical deformity—kyphosis or scoliosis, cervical spine fractures and dislocations, cervical spine infections or tumors. The fusion means joining several distinct vertebrae or motion segments together. A bone graft is placed around decorticated bone surfaces which incites a fracture healing-type response. This eventually leads to formation of bridging bone that connects the treated vertebrae together and eliminates any further motion, progressive instability or deformity.

With traditional spinal fusion with instrumentation the patient usually has two or more vertebrae joined into a single rigid structure. This surgery is performed to attempt to stop the motion of the diseased motion segment and additionally to correct spinal deformity. During the surgery the two vertebrae are held together with rods and screws, allowing for the bone to grow around the instrumentation and join the treated levels together.

The cervical fusion can be done from the front—anterior, from the back—posterior or combined—circumferential or 360 fusion. The posterior cervical fusion often accompanies a cervical laminectomy which destabilizes the cervical spine and may lead to a cervical kyphosis if not supplemented with a fusion and instrumentation. The placement of instrumentation greatly increases the chance of a successful fusion and obviates the need for external immobilization, such as a halo vest or a cervical orthosis. The ability to place cervical instrumentation safely and effectively is paramount for cervical spine surgeons.

The posterior instrumentation in the cervical spine is commonly accomplished with polyaxial screws placed in lateral masses of C1, pars, lamina or pedicles of C2, lateral masses or pedicles of C3-C7. The majority of cervical fixation is done in the subaxial cervical spine (below C2) with the use of lateral mass screws which are safe and easy to place and require minimal if any fluoroscopic guidance or navigation. The downside of lateral mass screws is a relatively poor bone purchase and a potential for a screw pullout or loosening, especially in patients with a significant osteoporosis or osteopenia. The same screw pullout can occur in a kyphotic deformity correction, instability such as in spine fractures, cervical dislocations or spinal osteotomy. In older patients the bone strength has deteriorated, and the screws have a higher chance of not holding within the bone due to a low push-in torque and pullout strength.

A cervical pedicle screw is a stronger alternative to a lateral mass screw. A cervical pedicle screw is feasible at C3-C6 levels, but very technically challenging due to the small size of the pedicles at those levels, the proximity to the transverse foramen and the possibility of a vertebral artery injury or a spinal cord injury.

An objective of the present invention is to provide spine surgeons with a spinal locking plate as an alternative to pedicle screws to help patients with spinal fusion surgery regardless of their bone condition and the extent of their spinal deformity. This plate is segmental, meaning that it is only fixed to one vertebra and does not bridge several vertebrae together. The present invention intends to provide users with a device that is designed to fixate on the densest regions of the vertebrae and gives the surgeon a safer and potentially stronger spinal anchor.

In order to accomplish that, a preferred embodiment of the present invention comprises a plate, a plurality of locking screws, a tulip assembly (monoaxial or polyaxial), a set screw and a rod assembly. Furthermore, the plurality of locking screws allows the present invention to securely attach to the spine of the patient without the need of engaging the full length of the pedicles and the vertebral body. Thus, the present invention is a spinal plate and screw system that provides patients of all ages and conditions with a secure spinal attachment over a larger footprint ensuring the fixation is safer and easier during the spinal fusion surgery.

### SUMMARY OF THE INVENTION

The present invention is a spinal plate for cervical application to facilitate a spinal fusion surgery. The present invention seeks to provide users with a device that can create a secure fixture on the spine without using longer invasive screws. In order to accomplish this the present invention comprises a plate that creates a footprint allowing for several small locking (screws that thread into the plate itself as well as the bone) and/or non-locking screws (screws that do not thread into the plate itself but only into the bone) to be placed. The plurality of locking screws ensures the plate is properly secured to the patient. Additionally, the polyaxial or monoaxial tulip assembly allows for an additional, adjustable axis of connection to the plate. Further, the rod assembly provides the user with a connection to another anchor along the spinal column-either another plate or a polyaxial lateral mass, pedicle, pars or laminar screw. Thus, the present invention is a spinal plate and screw system that provides a secure spinal attachment over a larger footprint ensuring that the fixation is safer and easier during the spinal fusion surgery.

### BRIEF DESCRIPTION OF THE DRAWINGS

FIG. 1 is a perspective view of the present invention.

FIG. 2 is an exploded view of one embodiment of the tulip assembly of the present invention.

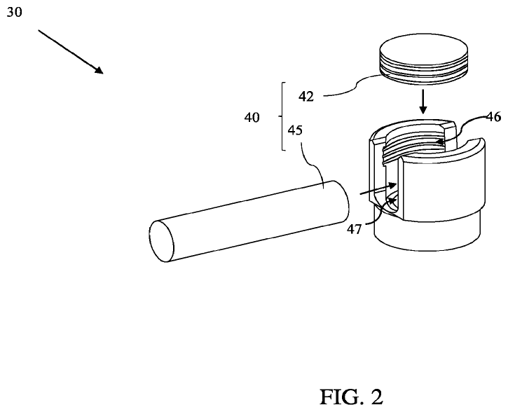

FIG. 3 is a side view of the present invention.

FIG. 4 is a top view of the present invention.

FIG. 5 is a bottom view of the present invention.

### DETAIL DESCRIPTIONS OF THE INVENTION

All illustrations of the drawings are for the purpose of describing selected versions of the present invention and are not intended to limit the scope of the present invention.

The present invention is a spinal plate (device) for cervical applications. An objective of the present invention is to provide users with a device to improve the spine fixation and to reduce the risk of nerve damage during spinal fusion surgery. The present invention intends to provide users with a device that is easier to apply. To accomplish this the present invention provides a device that comprises a plate 10, a plurality of locking screws 20, a tulip assembly 30, and a set screw 42.

The plurality of locking screws 20 and tulip assembly 30 attach to the plate 10 via the plurality of holes 15 placed on the plate 10. In some embodiments, the present invention may include a rod assembly 40 comprising a rod 45 and a screw 42 so that the rod 45 attaches perpendicularly to the tulip assembly 30 via the set screw 42. Thus, the present invention is a spinal plate and screw system that provides patients of all ages and conditions with a secure spinal attachment over a larger footprint ensuring the fixation is stronger, safer and easier for the patient during the spinal fusion surgery.

The present invention is used during spinal fusion surgery with the main plate component. The plate 10 is made of a lightweight metal material, for instance pure titanium, titanium alloy, cobalt-chrome, stainless steel, with small unique shape.

In its preferred embodiment the plate 10 comprises a plurality of threaded holes 15. The plate 10 can have either one, two, three, four, five, six, seven, eight, nine, ten or more threaded holes 15. The thread in the holes 15 is to accommodate the matching thread at the proximal (closer to the head) end of the locking screws 20.

The plate 10 may be flat or curved to accommodate the shape of the posterior elements of the spine. The curvature of the plate 10 or the bend of the plate 10 may be introduced pre-operatively during the plate manufacturing or intraoperatively using plate benders.

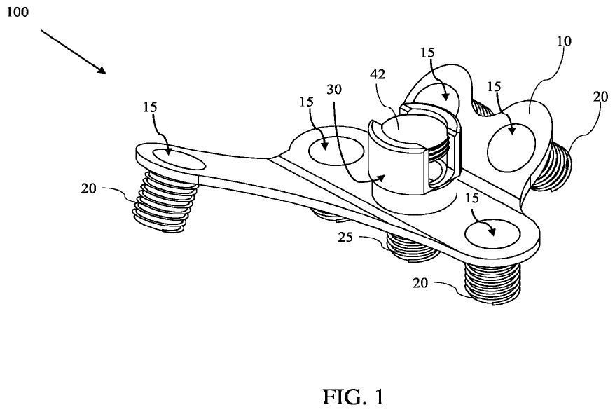

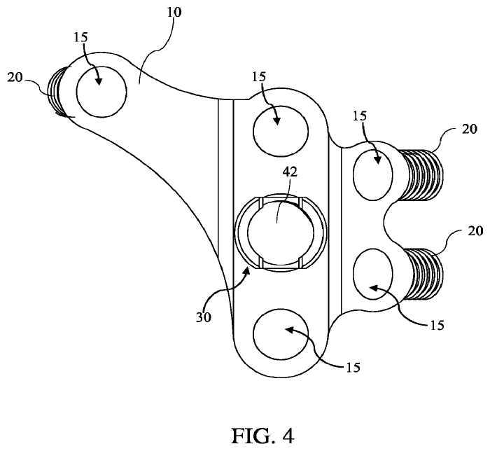

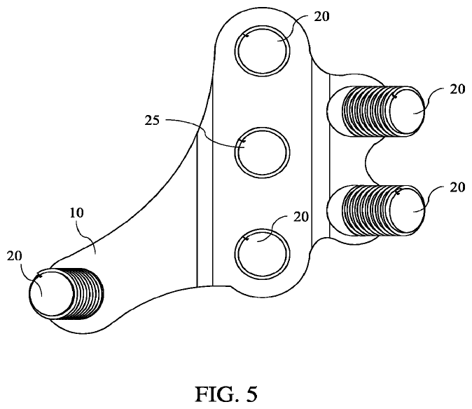

The plurality of holes 15 can be located around the edges of the plate 10 near the curved or straight portions as shown in FIGS. 1 and 3-5.

In some embodiments, the plurality of holes 15 is designed as circular or oblong threaded cutouts into the plate 10 that allow for a locking screw 20 to be placed within the open gap of the hole 15 without reducing the structural integrity of the plate 10.

The plate 10 allows for the present invention to be fixated on the patient on anatomy outside of the pedicle, which reduces the chance of a spinal cord or vascular injury.

In its preferred embodiment the plurality of holes 15 is designed with 4, 5 or 6 holes. It should be further noted that, the plate 10 can be created in many various shapes and sizes and plurality of holes could be positioned in various locations while still staying within the scope of the present invention.

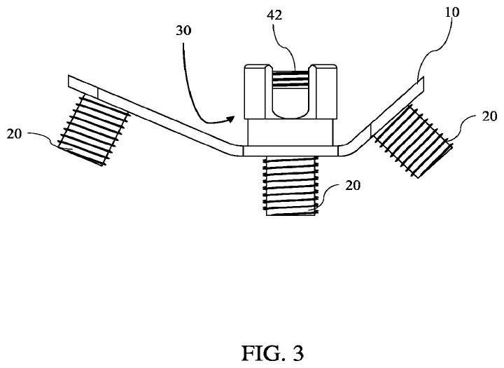

The plurality of locking screws 20 connects with the plate 10 via the plurality of threaded holes 15 on the plate 10 as shown in FIGS. 1 and 3. In its preferred embodiment, the plurality of locking screws 20 comprises a “star head”. However, the heads of the polyaxial screws 25 can have a “hexagonal head” for use with a torque-type screwdriver. The plurality of locking screws 20 is designed with a short length-typically 6-16 mm and a helix-shaped thread.

In some embodiments, the locking screws 20 may have a thread under the screwhead that matched the thread in the holes of the plate 10 and allows for the screws to be threaded into the plate 10 and create a fixed-angle construct.

The plurality of locking screws 20 ensure that that plate 10 can be attached to the spine over a larger footprint with 1, 2, 3, 4, 5, 6 or more locking screws 20. The shorter length of the plurality of locking screws 20 may ensure that neurological and vascular injury is minimized during the fixation. Additionally, the plurality of locking screws 20 is fastened into the plate 10, allowing the present invention to have a strong fixation onto the bone regardless of bone deterioration due to conditions such as osteoporosis.

The hex or star head of the screws (20, 25) can be designed as a divot at the top of the plurality of locking screws 20 to ensure the plurality of locking screws 20 can be rotated with an axially directed force.

The tulip/screw assembly 30 connects with the plate 10 via any of the plurality of holes 15. The tulip assembly 30 has a U-shape section with a circular cross section.

In its preferred embodiment the tulip assembly 30 comprises an inner thread 46 and a bottom hole 47. The inner thread 46 is positioned on the inner wall of the tulip assembly 30 and continues around the length of the tulip assembly 30 passing through the open sections inside of the tulip assembly 30. This design allows an additional screw to be tightened into place along the top side of the tulip assembly. Below the inner thread 46 is the bottom hole 47 that is positioned in the concentric center of the tulip assembly 30 circular cross-section.

In some embodiments, the bottom hole 47 allows for a locking screw 20 of the plurality of locking screws 20 to be threaded through the bottom hole 47 and the center hole of the plurality of holes 15 on the plate to attach the tulip assembly 30 to the plate 10 to the patient. Further, the size of the tulip assembly 30 can be adjusted to allow for multiple rod construction, in some embodiments, as well as having rods of different diameters all interconnected, increasing the rigidity and strength of the final construct.

In some embodiments, the present invention may include a locking polyaxial screw 25 and/or a locking monoaxial screw 25 to connect the tulip assembly 30 to the plate 10. In some other embodiments, the locking polyaxial screw 25 and/or the locking monoaxial screw 25 can also be used in any holes of the plate 10.

Generally, the polyaxial screw is used for connecting vertebrae to rods in spinal surgery, particularly spinal fusion procedures. The polyaxial screw has a spherical head enclosed in a housing, which allows the screw a range of motion along several different axes relative to the housing. The ball joint that may be included in the housing allows the surgeon some flexibility in placing the screws.

The monoaxial screw has no degree of freedom and the head is fixed just like a regular screw.

In one embodiment, the locking polyaxial screw 25 with a combination of the bone thread distally (to engage the vertebra) and the locking thread proximally (to lock into the plate) can be placed into any of the holes on the locking plate 10. The said screw can have a variable length, variable outer thread diameter and thread pitch and variable core diameter. The two key features of the said screw are the ability to lock into the plate (locking feature) and the ability to accept a rod to connect several elements together (polyaxial feature).

In one other embodiment, the locking monoaxial screw 25 with a combination of the bone thread distally (to engage the vertebra) and the locking thread proximally (to lock into the plate) can be similarly placed into any of the holes on the locking plate 10. The said screw can have a variable length, variable outer thread diameter and thread pitch and variable core diameter. The two key features of the said screw are the ability to lock into the plate (locking feature) and the ability to accept a rod into a rigid monoaxial tulip to connect several elements together.

The rod assembly 40 connects with the tulip assembly 30 via the inner thread 46. In its preferred embodiment the rod assembly 40 comprises a set screw 42 and a rod 45. The rod 45 is designed with a metal material that is placed within the tulip assembly 30 and reaches another vertebrae of the spine, allowing the present invention to be interconnected to other devices.

The rod 45 can be held into place with the set screw 42 that matches the thread of the inner thread 46 of the tulip assembly 30. The set screw 42 is rotated into the tulip assembly 30 creating a force on the rod 45 below inserted into the tulip assembly 30, ensuring it stays positioned properly.

With all the components working in tandem with each other it can be seen that the present invention is a spinal plate and screw system that provides patients of all ages and conditions with a secure spinal attachment over a larger footprint ensuring the fixation is safer and easier for the patient during the spinal fusion surgery.

Even though both locking plates 10 and polyaxial screws 25 have been used in orthopaedics for years, the main novelty of this invention is the coupling of the two via a locking polyaxial screw or locking monoaxial screw 25 that rigidly connects both the rod 45 and the plate 10.

Although the invention has been explained in relation to its preferred embodiment, it is to be understood that many other possible modifications and variations can be made without departing from the spirit and scope of the invention.

1. A device comprising:
a plate having a plurality of holes;
a plurality of locking screws attached to the plate;
a locking monoaxial screw attached to the plate;
a locking polyaxial screw;
a tulip assembly, having inner threads and a bottom hole, attached to the plate by the locking polyaxial screw;
a set screw having threads; and
a rod connected to the tulip assembly by the set screw.

2. The device as claimed in claim 1, wherein the plate includes one or more curved surfaces.

3. The device as claimed in claim 1, wherein at least one of the plurality of locking screws comprises a hex head.

4. The device as claimed in claim 1, wherein at least one of the plurality of locking screws includes a helix-shaped thread.

5. The device as claimed in claim 1, wherein at least one of the locking polyaxial screw and the monoaxial screw comprise a star head.

6. The device as claimed in claim 1, wherein the tulip assembly includes a U-shape section.

7. The device as claimed in claim 1, wherein the inner threads of the tulip assembly are positioned on an inner wall of the tulip assembly and continue around a length of the tulip assembly.

8. A device comprising
a plate with a plurality of holes, the plate having one or more curved surfaces;
a plurality of locking screws attached to the plate;
a locking polyaxial screw attached to the plate;
a monoaxial screw;
a tulip assembly, having inner threads and a bottom hole, attached to the plate by the monoaxial screw;
a set screw having threads; and a rod connected to the tulip assembly by the set screw.

9. The device as claimed in claim 8, wherein at least one of the plurality of locking screws comprises a hex head.

10. The device as claimed in claim 8, wherein at least one of the plurality of locking screws includes a helix-shaped thread.

11. The device as claimed in claim 8, wherein at least one of the locking polyaxial screw and the monoaxial screw comprise a star head.

12. The device as claimed in claim 8, wherein the tulip assembly includes a U-shape section.

13. The device as claimed in claim 8, wherein the inner threads of the tulip assembly are positioned on an inner wall of the tulip assembly and continue around a length of the tulip assembly.

14. A device comprising a plate having a plurality of holes;
a plurality of locking screws attached to the plate;
a monoaxial screw attached to the plate;
a locking polyaxial screw having a star head;
a tulip assembly, having inner threads a bottom hole, attached to the plate by the locking polyaxial screw;
a set screw having threads; and
a rod connected to the tulip assembly by the set screw.

15. The device as claimed in claim 14, wherein the plate includes one or more curved surfaces.

16. The device as claimed in claim 14, wherein at least one of the plurality of locking screws comprises a hex head.

17. The device as claimed in claim 14, wherein at least one of the plurality of locking screws includes a helix-shaped thread.

18. The device as claimed in claim 14, wherein the tulip assembly includes a U-shape section.

19. The device as claimed in claim 14, wherein the inner threads of the tulip assembly are positioned on an inner wall of the tulip assembly and continue around a length of the tulip assembly.

20. The device as claimed in claim 14, further includes one or more additional rods connected to the tulip assembly by the set screw.
